(19) United States
(12) Patent Application Publication
(10) Pub. No.: US 2026/0144507 A1
(43) Pub. Date: May 28, 2026

(54) SYSTEMS AND METHODS FOR VISUALIZING COMPUTED TOMOGRAPHY ANGIOGRAPHY IMAGES

(71) Applicant: The Regents of the University of California, Oakland, CA (US)

(72) Inventors: Nadir Weibel, La Jolla, CA (US); Brett Meyer, San Diego, CA (US); Weichen Liu, San Diego, CA (US); Jacob Lin, Arcadia, CA (US)

(21) Appl. No.: 19/347,276

(22) Filed: Oct. 1, 2025

Related U.S. Application Data
(60) Provisional application No. 63/725,191, filed on Nov. 26, 2024.

(51) Int. Cl. A61B 6/46; A61B 6/03; A61B 6/50; G06F 3/01; G06T 7/00; G06T 15/08; G06T 15/20; G06T 15/30; G06T 19/00

(52) U.S. Cl. CPC A61B 6/465; A61B 6/032; A61B 6/463; A61B 6/466; A61B 6/504; G06F 3/017; G06T 7/0012; G06T 15/08; G06T 15/20; G06T 15/30; G06T 19/00; G06T 2200/24; G06T 2207/10081; G06T 2207/20092; G06T 2207/30008; G06T 2207/30101; G06T 2210/41; G06T 2210/62

(57) ABSTRACT

Embodiments herein rapidly transform computed tomography angiography (CTA) data into an interactive, holographic visualization for clinical use. In one embodiment, a system for visualizing computed tomography angiography (CTA) images comprises a data interface configured to receive CTA image data in a Digital Imaging and Communications in Medicine (DICOM) format. The system also comprises a processor configured to render a plurality of two-dimensional CTA slice images into a three-dimensional (3D) volumetric dataset from the CTA image data, to filter the 3D volumetric dataset to resolve vascular structures relative to surrounding tissue and bone, and to generate an extended reality (XR) holographic representation of the filtered 3D volumetric dataset. The system also comprises an XR display device configured to present the holographic representation to a user for the user to manipulate the holographic representation and identify a vascular abnormality.

### CROSS REFERENCE TO RELATED APPLICATIONS

This patent application claims priority to, and thus the benefit of an earlier effective filing date from, U.S. Provisional Patent Application No. 63/725,191 (filed Nov. 26, 2024), the contents of which are hereby incorporated by reference.

### FIELD

The present invention relates generally to medical imaging systems, and more particularly to systems and methods for visualizing computed tomography angiography (CTA) images using extended reality (XR).

### BACKGROUND

Computed tomography angiography (CTA) is a widely used imaging modality for evaluating blood vessels in the brain and other regions of the body. A typical CTA scan generates hundreds of two-dimensional (2D) slice images in a Digital Imaging and Communications in Medicine (DICOM) format. Physicians interpret these images by scrolling through the slices or by reviewing planar reformats such as axial, sagittal, and coronal views.

In some cases, specialized workstations may reconstruct a three-dimensional (3D) model or maximum intensity projection (MIP) from the 2D slices. However, these reconstructions are typically displayed on conventional two-dimensional monitors. As a result, while the user can rotate or scroll through the 3D dataset on screen, the visualization itself remains flattened into two dimensions, which can limit depth perception and increase cognitive load when evaluating vascular anatomy. The interpretation of CTA studies can therefore be time-consuming and cognitively demanding. Physicians must mentally reconstruct the vascular anatomy by integrating information across multiple slices. Subtle vascular abnormalities, such as large vessel occlusions, can be overlooked, particularly by less experienced readers. Delays or inaccuracies in CTA interpretation can negatively impact patient outcomes, especially in emergent conditions where every minute is critical. Additionally, previous 3D reconstructions required significant post-processing time, which is not practical in acute care settings such as stroke evaluation

Recent advances in visualization technologies, including augmented reality and virtual reality, have demonstrated potential to improve medical imaging interpretation by enabling immersive and interactive review of 3D datasets. However, existing systems are not optimized for rapid, on-the-fly volumetric reconstruction of CTA data into holographic representations suitable for real-time clinical decision-making.

Accordingly, there remains a need for a system that can automatically render CTA DICOM slice data into a 3D volumetric dataset, filter the dataset to resolve vascular structures relative to surrounding tissue and bone, and present an interactive holographic representation in extended reality. Such a system would allow clinicians to rapidly manipulate vascular holograms, reduce interpretation time, improve detection of vascular abnormalities, and facilitate timely clinical intervention.

### SUMMARY

The embodiments described herein provide for systems and methods to rapidly transform computed tomography angiography (CTA) data into an interactive, holographic visualization for clinical use. A data interface imports CTA slice images in DICOM format, and a processing system (e.g., employing one or more graphics processing units, or “GPUs”) renders the plurality of two-dimensional slices into a three-dimensional volumetric dataset. The system further filters the dataset to distinguish vascular structures from surrounding bone and soft tissue, and then generates an artificial reality holographic representation. This hologram is displayed on an artificial reality, enabling clinicians to view vasculature in true 3D spatial context rather than on a conventional 2D monitor.

The hologram may be manipulated through gesture detection to rotate, resize, clip, or probe the vasculature in real-time. The system incorporates vessel-specific visualization tools such as Clip-Sphere/Clip-Box for bone removal, probe spotlighting, and preset transfer functions to emphasize vascular structures. By automating volumetric reconstruction and optimizing rendering through GPU acceleration, the system generates clinically usable holograms within about one minute (e.g., 10-30 seconds). The resulting system improves accuracy and speed of detecting vascular abnormalities such as large vessel occlusions, enabling faster clinical decisions in acute stroke care.

In one embodiment, a system for visualizing computed tomography angiography (CTA) images comprises a data interface configured to receive CTA image data in a Digital Imaging and Communications in Medicine (DICOM) format, and a processor (e.g., one or more graphical processing units, or “GPUs”) configured to render a plurality of two-dimensional CTA slice images into a three-dimensional (3D) volumetric dataset from the CTA image data, to filter the 3D volumetric dataset to resolve vascular structures relative to surrounding tissue and bone, and to generate an extended reality (XR) holographic representation of the filtered 3D volumetric dataset. The system also includes an XR display device configured to present the holographic representation to a user for the user to manipulate the holographic representation and identify a vascular abnormality (e.g., to facilitate detection of a large vessel occlusion in a cerebral artery). The XR display device may include one or more camera modules configured to detect gestures by the user to manipulate the holographic representation. The XR display device may be further configured to present at least one holographic radiology image pinned in a spatial location relative to the holographic representation of the 3D volumetric dataset.

In some embodiments, the processor is further configured to filter the 3D volumetric dataset with a clipping tool to remove bone elements from the volumetric dataset to expose vascular structures. The processor may be further configured to spotlight one or more vessels within the holographic representation with a probe implemented by a gesture of the user. The processor may be further configured to apply one or more preset transfer functions to emphasize vascular structures relative to bone or soft tissue.

In some embodiments, the processor is further configured to render the 3D volumetric dataset in substantially real-time, the rendering being completed within about one minute of receipt of the CTA image data. The processor may be further configured to allow the user to adjust a transparency level of non-vascular structures within the 3D volumetric dataset. The processor may be further configured to generate the holographic representation with one or more preset anatomical views including at least one of an axial orientation, a coronal orientation, or a sagittal orientation.

In another embodiment, a method for visualizing computed tomography angiography (CTA) images comprises receiving CTA image data in a Digital Imaging and Communications in Medicine (DICOM) format, rendering a plurality of two-dimensional CTA slice images into a three-dimensional (3D) volumetric dataset from the CTA image data, filtering the 3D volumetric dataset to resolve vascular structures relative to surrounding tissue and bone, generating an extended reality (XR) holographic representation of the filtered 3D volumetric dataset, presenting the holographic representation to a user via an XR display device configured to for the user to manipulate the holographic representation and identify a vascular abnormality.

In another embodiment, a non-transitory computer readable medium comprises instructions that, when executed by a processing system, are operable to direct the processing system to visualize computed tomography angiography (CTA) images. The instructions further direct the processing system to receive CTA image data in a Digital Imaging and Communications in Medicine (DICOM) format, render a plurality of two-dimensional CTA slice images into a three-dimensional (3D) volumetric dataset from the CTA image data, filter the 3D volumetric dataset to resolve vascular structures relative to surrounding tissue and bone, generate an extended reality (XR) holographic representation of the filtered 3D volumetric dataset, and present the holographic representation to a user via an XR display device configured to for the user to manipulate the holographic representation and identify a vascular abnormality.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 illustrates an exemplary system for visualizing computed tomography angiography (CTA) images by generating a holographic representation suitable for display in extended reality.

FIG. 2 illustrates a method for visualizing CTA images and presenting them in extended reality, in one exemplary embodiment.

FIG. 3 illustrates a user interacting with the holographic representation in an extended reality environment, in one exemplary embodiment.

FIG. 4 shows a block diagram of an exemplary head-mounted display (HMD) system that may be configured to present virtual scenes to a user, in one exemplary embodiment.

FIG. 5 illustrates a head-mounted augmented reality display device 500, in one exemplary embodiment.

FIG. 6 illustrates a head-mounted display device 500, in one exemplary embodiment.

FIG. 7 depicts one illustrative cloud computing system 600 operable to perform the above operations by executing programmed instructions tangibly embodied on one or more computer readable storage mediums.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

The embodiments of the instant disclosure may include or be implemented in conjunction with various types of artificial reality systems. Artificial reality is a form of reality that has been adjusted in some manner before presentation to a user, which may include, e.g., a virtual reality (VR), an augmented reality (AR), a mixed reality (MR), or some combination thereof. Artificial reality content may include completely generated content or generated content combined with captured (e.g., real-world) content. The artificial reality content may include video, audio, haptic feedback, or some combination thereof, any of which may be presented in a single channel or in multiple channels, such as stereo video that produces a three-dimensional effect to the viewer. As used herein, extended reality (XR) is a term that is intended to encompass all forms of artificial reality.

Artificial reality systems may be implemented in a variety of different form factors and configurations. Some artificial reality systems may be designed to work with a head-mounted display (HMD). In artificial reality, an HMD device may partially or completely obstruct the user's view of the real-world environment. Depending on the devices, the user may see all or a portion of the user's surroundings. Thus, as part of a training phase, in some embodiments, the user may first be prompted to visualize the real-world environment with the HMD device, which can generate a model of that environment. During an interaction or operational phase, the user may interact with a virtual environment, such that the movement of a user from one location to another in the virtual environment is accomplished by the user moving within the real-world environment. For example, the user may move objects within the virtual environment being presented in a display of the HMD device the user is wearing. To provide the user with awareness of the real-world environment during the interaction phase, a portion of the model generated during the training phase may be shown to the user in a display along with the virtual scene or environment.

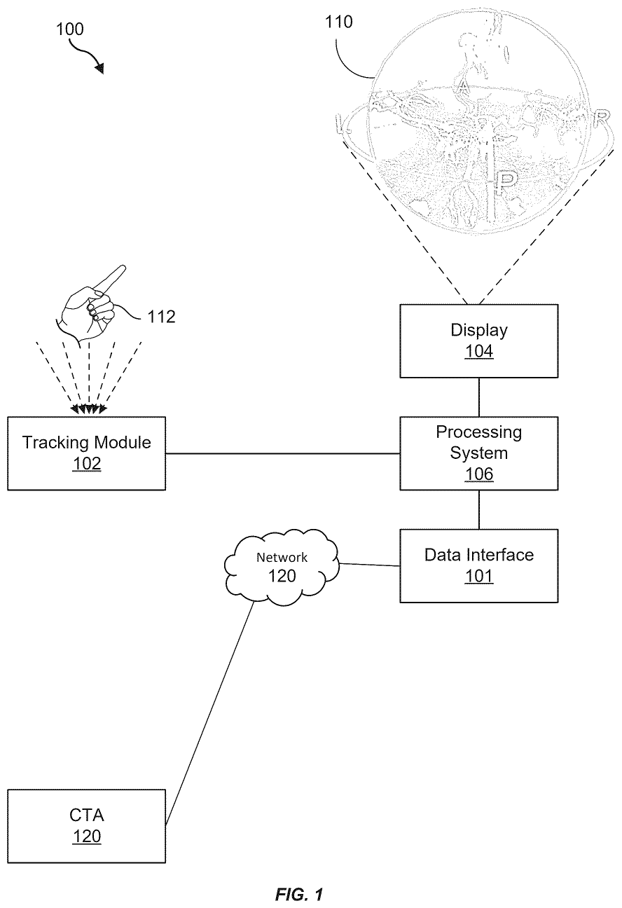

FIG. 1 illustrates an exemplary system 100 for visualizing computed tomography angiography (CTA) images by generating a holographic representation 110 suitable for display in extended reality. The system includes a data interface 101, a processing system 106, a tracking module 102, and a display 104. Together, these components operate to generate and present a holographic representation 110 of vascular structures reconstructed from CTA image data, which may be manipulated by a user through gestures 112. For example, the system 100 is configured to receive CTA image data 120, process the data into a volumetric representation, and present a hologram that can be manipulated by a user through gestures 112. Communication between components may occur through a network 120, which may be a wired or wireless communication link.

The data interface 101 is configured to receive CTA imaging data 120 from an external source, such as a CT scanner or a picture archiving and communication system. The CTA data is generally formatted according to the Digital Imaging and Communications in Medicine (DICOM) standard and comprises a series of thin slice images spanning a patient's head or neck. The data interface manages transport of the DICOM files across the network 120 and ensures proper ordering and orientation of the slice series before delivery to the processing system 106. In some embodiments, the data interface is in communication with a picture archiving and communication system (PACS) of a hospital or a CT scanner console and is implemented through wired or wireless communication modules.

The processing system 106 receives the CTA data through the data interface 101 and constructs a three-dimensional volumetric dataset by interpolating the series of two-dimensional slices into a voxel-based grid. The processing system 100 includes memory for storing the CTA dataset, intermediate volumetric reconstructions, and executable instructions for rendering and visualization. Filtering operations may then be applied to the volumetric dataset in order to resolve vascular structures relative to surrounding tissue and bone. In some embodiments, the processing system 106 applies transfer functions that adjust voxel brightness and opacity to emphasize vessels and suppress non-vascular structures. The processing system 100 may also execute bone removal algorithms, clipping operations, and probe tools that allow selective spotlighting of vessels within the hologram. To achieve real-time responsiveness, the processing system 106 may incorporate one or more graphics processing units configured to carry out rendering and filtering computations in parallel.

The holographic representation 110 generated by the processing system 106 is transmitted to the display 104, an XR display device capable of presenting three-dimensional images anchored in physical space. The display 104 renders the holographic representation in the user's environment such that the user perceives the vasculature as a spatially manipulable object.

The tracking module 102 is configured to detect user gestures 112 and transmit corresponding input signals to the processing system 106. The tracking module may include optical or infrared cameras and depth sensors integrated with the display 104 or positioned externally. These sensors capture image and depth data of the user's hands, which is converted into skeletal models representing fingertip positions, palm orientation, and finger articulation.

The skeletal data is streamed to the processing system 106, which classifies the data into discrete gestures using pattern recognition algorithms or trained machine learning models. Recognized gestures are mapped to commands that manipulate the holographic representation 110. For example, a pinch gesture may be interpreted as a grab that allows the user to rotate the hologram, a spreading of two fingers may be interpreted as a zoom to scale the hologram, and a pointing gesture may activate a probe function that highlights a vessel intersected by the gesture path. The processing system 106 then applies the corresponding command to the volumetric dataset and updates the holographic representation 110.

Through this interaction loop, CTA imaging data 120 is converted into a volumetric holographic representation 110 and presented on the display 104, while the tracking module 102 detects and communicates gestures 112 that allow the user to explore the hologram in real-time. The system thereby enables a clinician to examine the vascular system of the brain more rapidly and accurately than by reviewing two-dimensional slices on conventional monitors, improving detection of abnormalities such as large vessel occlusions.

The display device 104 presents the holographic representation 110 of the filtered volumetric dataset in the user's physical environment. In one embodiment, the display device 104 is a head-mounted augmented reality headset that provides stereoscopic images of the hologram. The holographic representation 110 appears to be anchored in space, allowing the user to move around it and view the vascular anatomy from multiple perspectives. The display device 104 operates in conjunction with the tracking module 102, so that the hologram responds in real-time to gestures performed by the user.

The holographic representation 110 depicts the vasculature of the patient reconstructed from the CTA data. The hologram may include cerebral arteries and carotid vessels rendered with sufficient detail to permit detection of large vessel occlusions or other abnormalities. As the user performs gestures 112, the processor modifies the hologram by rotating it, scaling it, clipping away non-vascular structures, or applying probe and spotlighting functions. The updated hologram is then presented to the user without perceptible delay, creating a closed-loop system where CTA data is transformed into a manipulable three-dimensional visualization of the vascular system of the brain.

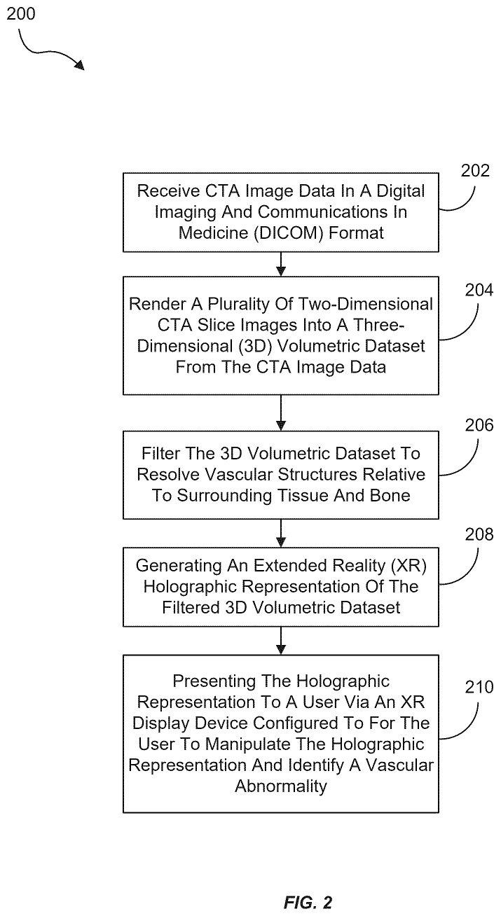

FIG. 2 illustrates a method 200 for visualizing computed tomography angiography images and presenting them in extended reality. The method begins at step 202, where CTA image data is received in a Digital Imaging and Communications in Medicine (DICOM) format. The received data typically comprises hundreds of two-dimensional slices generated by a CT scanner, each slice representing a thin cross-section of the patient's vasculature. At step 204, the processor renders the plurality of two-dimensional slice images into a three-dimensional volumetric dataset, interpolating voxel values across the slices to create a continuous volumetric grid. At step 206, the volumetric dataset is filtered to resolve vascular structures relative to surrounding tissue and bone. This filtering may include applying transfer functions to enhance contrast, removing bone using clipping algorithms, or suppressing non-vascular tissue through opacity modulation. At step 208, the processor generates a holographic representation of the filtered dataset suitable for presentation in an XR environment. And, at step 210, the holographic representation is displayed to a user via an XR headset, allowing the user to manipulate the holographic image through gestures and identify vascular abnormalities in real-Attorney time. The flow of FIG. 2 highlights the end-to-end automation of the system, from receipt of raw CTA data to immersive exploration of vascular anatomy.

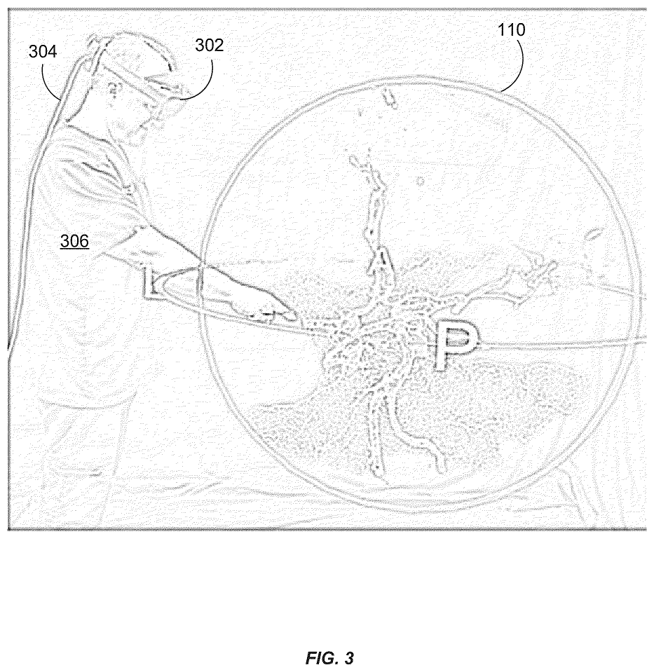

FIG. 3 illustrates a user 306 interacting with the holographic representation 110 in an XR environment. In this embodiment, the user is equipped with an augmented reality display device 302, which may be a head-mounted headset tethered by a connection 304 to a computing unit that executes rendering and filtering processes. The holographic representation 110 is projected into the user's field of view as a spatial object anchored in real-world space. The vascular anatomy can be explored by the user through natural hand gestures without the need for external controllers. For example, the user may rotate the holographic brain vasculature by moving a hand laterally, zoom into particular vessels by performing a pinch-and-spread motion, or direct a probe function by pointing to a specific vessel segment. The augmented reality system tracks the user's head and hand positions, synchronizing the holographic rendering with physical movements to maintain immersion and interactivity. In this manner, FIG. 3 demonstrates how the system extends traditional 2D interpretation of CTA images into an intuitive 3D workspace where vascular structures can be manipulated as though they were physically present.

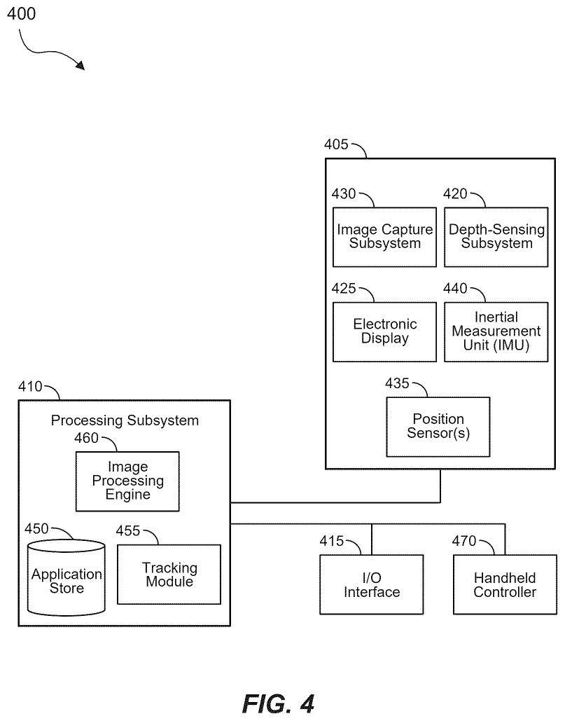

FIG. 4 shows a block diagram of an exemplary head-mounted display (HMD) system 400 that may be configured to present virtual scenes, such as captured environments, artificially generated content, or combinations thereof, to a user. The HMD system 400 may operate in a virtual reality environment, an augmented reality environment, a mixed reality environment, or any combination of the foregoing. In the embodiment illustrated, the HMD system 400 includes an HMD device 405 that communicates with a processing subsystem 410 and an input/output (I/O) interface 415. The HMD device 405 may be implemented to fully obstruct the user's view of the real-world environment in some configurations, while in others the device only partially obstructs the user's view or selectively obstructs the view depending on the content presented.

Although FIG. 4 depicts a single HMD device 405 and a single I/O interface 415, other embodiments may include multiple HMD devices, each with an associated I/O interface, all connected to a shared processing subsystem 410. In some embodiments, the processing subsystem 410 may be integrated into the HMD device 405, while in others the subsystem may be separate and communicate with the device through a wired or wireless link.

The HMD device 405 is configured to present a variety of content to the user. This content may include artificially rendered virtual-world environments or augmented views of the real-world environment overlaid with computer-generated elements such as two-dimensional or three-dimensional imagery, video, or sound. Audio may be provided through speakers or headphones, which may be internal to the HMD device or externally coupled. In some embodiments, the speakers or headphones are detachable from the HMD housing. The HMD device may include one or more bodies that are coupled together, with rigid couplings causing the bodies to move as a single unit, and non-rigid couplings permitting relative movement.

In some embodiments, the HMD device 405 incorporates a depth-sensing subsystem 420, an electronic display 425, an image capture subsystem 430, one or more position sensors 435, and an inertial measurement unit (IMU) 440. These components may collectively provide a positioning subsystem that establishes the location and orientation of the HMD device with respect to the surrounding environment. In certain embodiments, the HMD device may also include eye-tracking and/or gaze estimation systems that determine the direction of the user's gaze. The specific components included in a given HMD device may vary depending on the configuration.

The depth-sensing subsystem 420 is configured to capture data describing distances within the local area around the HMD device. In some embodiments, the subsystem computes depth maps directly by applying computer vision techniques, structured light processing, time-of-flight imaging, or simultaneous localization and mapping (SLAM). In other embodiments, the subsystem transmits raw data to the processing subsystem 410, which constructs depth maps from the information. These depth maps may be used to generate a model of the local environment, and the subsystem may therefore also be referred to as a localization and modeling subsystem.

The electronic display 425 presents images to the user based on content provided by the processing subsystem 410. In some embodiments, the display is implemented as a single screen, while in others multiple displays are included, one for each eye of the user. Examples of display technology include liquid crystal displays, organic light-emitting diode displays, active-matrix organic light-emitting diode displays, and transparent organic light-emitting diode displays. In some embodiments, the display may be opaque so that the user cannot directly view the surrounding environment through the panel.

The image capture subsystem 430 includes one or more cameras that capture still or video images of the environment surrounding the HMD device. These cameras may provide stereoscopic views that are processed to generate information describing the environment or the position of the HMD device within the environment. In some cases, wide-angle lenses or SLAM cameras are employed to capture an expanded field of view. The captured images may be processed by the image capture subsystem itself or by the processing subsystem 410 to generate a three-dimensional model of the local environment.

The processing subsystem 410 may further process images captured by the image capture subsystem 430 to extract details of the environment, such as color, pattern, or texture. These aspects may be stored in a database and associated with portions of the environment model, so that subsequent renderings reproduce the visual appearance of real-world features such as walls, floors, or furnishings.

The inertial measurement unit 440, in cooperation with one or more position sensors 435, generates signals describing the motion and orientation of the HMD device. The position sensors may include accelerometers, gyroscopes, magnetometers, or other motion-detecting elements. The IMU integrates the signals to determine an estimated position, orientation, or velocity of the HMD device relative to an initial reference point. This information may be used to define a personal zone representing the space occupied by the user within the environment.

The I/O interface 415 enables a user to send action requests and receive responses from the processing subsystem 410 or from a handheld controller 470. The I/O interface may accommodate multiple input devices, including keyboards, mice, gloves, or bracelets, and may support one or more handheld controllers. The handheld controller 470 may itself include an IMU for position tracking and may be capable of generating haptic feedback to the user in response to system events.

The processing subsystem 410 provides content to the HMD device 405 based on data received from the various sensors and interfaces. In one embodiment, the processing subsystem includes an image processing engine 460 and a tracking module 455. The image processing engine may generate or update rendered content for presentation, while the tracking module calibrates and refines positional information to maintain accurate tracking of the HMD device and any handheld controllers. Calibration may include adjustment of focus parameters of the depth-sensing subsystem or correction based on signals from the IMU. If tracking is lost or degraded, the tracking module may perform recalibration.

The tracking module 455 may also predict future positions and orientations of the HMD device to reduce latency in updating displayed content. It may further track features such as the user's hands or eyes, generating proxies that represent the volume occupied by the user or the direction of gaze. This information can be combined with sensor data to enhance realism and interactivity of the virtual or augmented content.

The image processing engine 460 may generate a three-dimensional model of the local environment based on depth information, structured light patterns, or other collected data. The engine may update the model as the environment changes and may extract visual characteristics to ensure accurate rendering of scenes. In addition, the image processing engine may integrate user actions detected by the I/O interface or handheld controller with application logic, generating visual, audio, or haptic feedback to the user to confirm execution of commands.

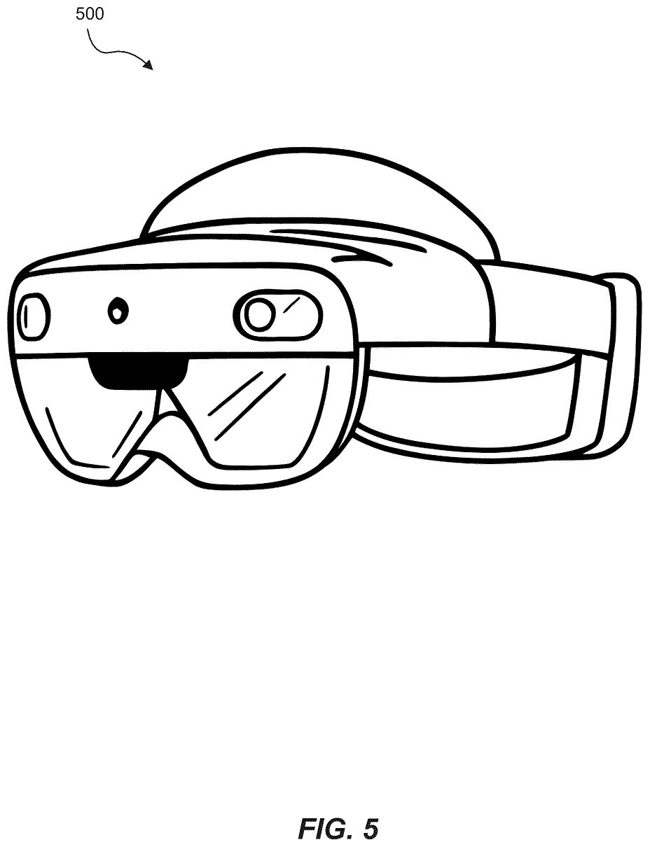

FIG. 5 illustrates a head-mounted augmented reality display device 500, in one exemplary embodiment. The augmented reality device 500 comprises a visor portion housing an array of optical waveguides and sensors configured to project virtual imagery into the user's field of view while maintaining transparency for real-world observation. A plurality of outward-facing cameras and depth sensors capture environmental features to enable simultaneous localization and mapping (SLAM), thereby permitting stable anchoring of virtual objects within the physical environment. The device further includes an adjustable headband to secure the visor on the user's head and integrated processing circuitry to fuse sensor data, manage graphical rendering, and provide real-time interaction between the user and computer-generated content.

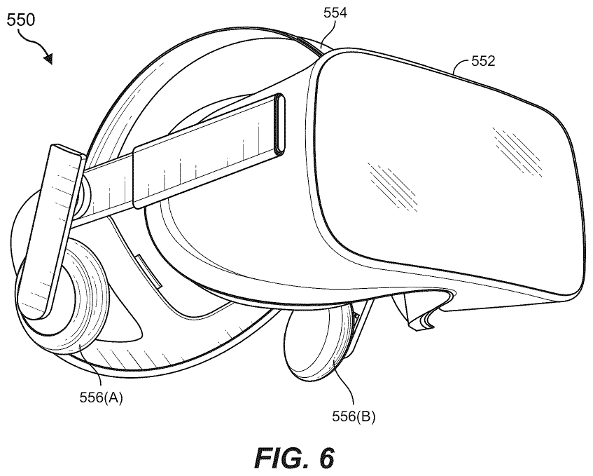

FIG. 6 illustrates one embodiment of a head-mounted display device 550 that may be used with the system described herein. The device 550 includes a housing 552 supporting an electronic display 554 positioned to present stereoscopic content to the user. The housing may be secured to the user's head by adjustable straps 556(A) and 556(B), which ensure proper alignment of the optics and display. The HMD 550 may integrate headphones or speakers for presenting audio information, such as system alerts or voice communication during collaborative review of CTA images. The display 504 may be implemented as an OLED, LCD, or other high-resolution panel suitable for rendering the fine vascular detail required for clinical diagnosis. In some embodiments, the HMD device 550 communicates with an external processing subsystem, while in other embodiments, processing components are embedded directly within the housing. The physical design of the HMD ensures comfort during extended use, optical clarity for accurate depth perception, and precise presentation of holographic vascular models derived from CTA data.

In some embodiments, methods can include using this system to improve the telemedicine interaction with patients. For example, incorporating the Holo-Stroke-CTA Hologram into telemedicine discussions with patients (either in Tele-stroke or non-telestroke telemedicine interactions with patients), would allow the provider to show the patient's specific DICOM images to the patient via hologram, to increase patient understanding of the vessels and their abnormalities.

In some embodiments, the system can be used in hologram education and training. For example, due to the ability to visualize in three dimensions the vasculature, this system can be used in the standard education of providers (e.g., including but not limited to physicians, physician in training, nurses, nurses in training, and technicians and technicians in training) to better understand and diagnosis vessel, tissue, and bony abnormalities. Example provider specialties include but are not limited to vascular neurology, neurosurgery, radiology, and neuroradiology. In its current embodiment, and with appropriate HIPAA safeguards in place, the system is able to access DICOM images stored on PACs servers, for these providers and trainees to review.

In some embodiments, the system can be used in Transcranial Doppler Assessments (TCD). For example, due to the ability to visualize in three dimensions the vasculature, this system can be used to augment TCD assessments. In one embodiment of this system, providers can align the 3D vessel representation to known landmarks on a patient's head to result a coordinate specific representation of the patient's vasculature while performing a TCD. Adding a 3D probe (e.g., with depth features marked) can assist the ultra sonographer in not only placing the TCD probe and aligning it to the correct course of the vessel, but the system can then automatically adjust/move more proximally and distally (e.g., using optimization of vessel waveform to guide probe placement) to appropriately map the vessels and result TCD waveforms at various depts within the vessel course.

In some embodiments, the system can be used in Real-Time LVO interventional Augmentation. For example, due to the ability to visualize in three dimensions the vasculature, this system can be used to augment standard angiogram procedures. In one embodiment of this system, providers can align the 3D vessel representation to known landmarks on a patient's head to result a coordinate specific representation of the patient's vasculature while performing an angiogram (diagnostic or therapeutic). Registering a virtual catheter tip to a standard angiogram catheter prior to groin puncture insertion, will allow co-registration and an ability to visualize the catheter as it traverses through the vasculature to the LVO lesion (or other lesions). Adding this co-registration can assist the neuro-interventionalist in performing the procedure/guiding the catheter to its appropriate location.

In some embodiments, the system can be used in Artificial Intelligence Perfusion vs. Core Overlays. For example, due to the ability to render DICOM images in 3D space, the tool allows for rendering of the vascular structures, as well as any secondary DICOM images showing overlay of perfusion and core illustrations (e.g., such as is often outputted from various AI software tools currently in use for assessment of perfusion/core mismatch in patients with large vessel occlusions.

In some embodiments, the system can be used in MeVOs. For example, the system can be used not just to analyze and evaluate Large Vessel Occlusions (LVOs) but also to evaluate Medium Vessel Occlusions (MeVOs), and more distal vessel occlusions.

In some embodiments, the system can be used in calcific atherosclerosis. For example, due to the ability to differentiate osseous structures and calcified objects (e.g., such as intracranial calcifications), the system can be used to differentiate presence of atherosclerosis and vessel narrowing due to different cases (e.g., the presence or absence of calcification in the vasculature)

In some embodiments, the system can be used in acute vs. chronic occlusions: For example, due to the ability to visualize in three dimensions the endings of vessels, there is an ability to differentiate chronic occlusions (e.g., usually with tapered endings of vessels) from more acute occlusions (e.g., usually with a more abrupt ending with a meniscus sign often present at the distal cutoff location).

In some embodiments, the system can be used in stenoses vs. sub-occlusive thromboses. For example, due to the ability to visualize in three dimensions the endings of vessels, there is an ability to differentiate high grade stenoses from acute sub-occlusive thromboses.

In some embodiments, the system can be used in intra-osseous vasculature. For example, due to the ability to use the Clip-Box tool to create a 3D visualization of planes of section throughout the volume rendering, the user can use Clip-box to slice through bone and assess vasculature even along its intra-osseous course. This can be important to assess for vessel stenoses along an intraosseous course of the vessel as it traverses different bony landmarks.

In some embodiments, the system can be used in LVO interventional planning. For example, due to the ability to visualize vessel contours in three dimensions, there is an ability to assess for intracranial LVOs. This can additionally facilitate a neurointerventionalist's view of the 3D vascular anatomy to guide decisions about selecting appropriate catheters, thrombectomy devices, and other techniques.

In some embodiments, the system can be used in intracranial aneurysms and AVMs. For example, due to the ability to visualize vessel contours in three dimensions, there is an ability to assess for intracranial aneurysms and AVMs. This can allow for a deeper understanding of the 3D nature of the vascular anomaly and guide interventional treatment strategies.

In some embodiments, the system can be used in extra-cranial stenoses. For example, due to the ability to visualize vessel contours in three dimensions, there is an ability to assess for high grade stenoses of vessels in the neck (e.g., Internal carotid artery or vertebral artery).

In some embodiments, the system can be used in cardiac stenoses. For example, due to the ability to visualize vessel contours in three dimensions, there is an ability to assess for high grade stenoses of cardiac vessels.

In some embodiments, the system can be used in aortic abnormalities. For example, due to the ability to visualize vessel contours in three dimensions, there is an ability to assess for abnormalities of the chest or abdominal aorta (e.g., including but not limited to aneurysms of the ascending or descending aorta).

In some embodiments, the system can be used in orthopedic abnormalities. For example, due to the ability to visualize bony contours in three dimensions, there is an ability to assess for orthopedic abnormalities throughout the body (e.g., including but not limited to fractures, bony masses, and other bone-based lesions).

In some embodiments, the system can be used in venous abnormalities. For example, due to the ability to modify transfer function focus to arteries or veins, the tool can preferentially assess the venous system (as opposed to the arterial system). This can be helpful to assess for venous occlusions in intra/extracranial sinuses or veins.

In some embodiments, the system can be used in CT bone visualization. For example, due to the ability to modify transfer function focus to non-vascular structures (e.g., and more on bony landmarks), the system can preferentially assess the bony structure to assess for osseous abnormalities. This can be usual for assessing mass lesions with different imaging characteristics which can be differentiated by setting a different transfer function curve value.

In some embodiments, the system can be used in CT tissue visualization. For example, due to the ability to modify transfer function focus to non-vascular structures (and more on tissue specific structures), the system can preferentially assess the tissue structure to assess for parenchymal abnormalities. This can be usual for assessing for tumor or other mass lesions with different imaging characteristics which can be differentiated by setting a different transfer function curve value.

In some embodiments, the system can be used in MRI bone visualization. For example, due to the ability to modify transfer function focus to non-vascular structures (e.g., and more on bony landmarks), the tool can preferentially assess the bony structure to assess for osseous abnormalities. This can be usual for assessing mass lesions with different imaging characteristics which can be differentiated by setting a different transfer function curve value.

In some embodiments, the system can be used in MRI Tissue Visualization. For example, due to the ability to modify transfer function focus to non-vascular structures (and more on tissue specific structures), the system can preferentially assess the tissue structure to assess for parenchymal abnormalities. This can be usual for assessing for tumor or other mass lesions with different imaging characteristics which can be differentiated by setting a different transfer function curve value.

In some embodiments, the system can be used in MRA vessel visualization. For example, due to the ability to modify transfer function focus to vascular structures in non-CTA scans such as DICOM MRI imaging, the system can preferentially assess the vascular structures as visualized by MRA technique. This can be usual for assessing for vascular lesions (including but not limited to LVOs, MeVOs, Aneurysms, AVMs, etc.) with different imaging characteristics which can be differentiated by setting a different transfer function curve value.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as health server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by an entity. Cloud computing relies on the sharing of resources, and generally includes on-demand self-service, broad network access, resource pooling, rapid elasticity, and measured service.

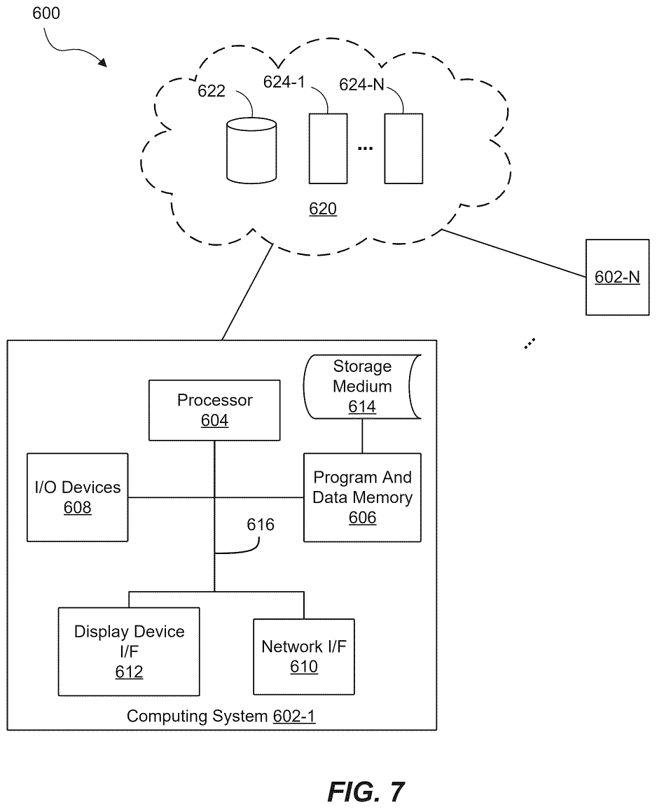

FIG. 7 depicts one illustrative cloud computing system 600 operable to perform the above operations by executing programmed instructions tangibly embodied on one or more computer readable storage mediums. The cloud computing system 600 generally includes the use of a network of remote servers hosted on the internet to store, manage, and process data, rather than a local server or a personal computer (e.g., in the computing systems 602-1-602-N). Cloud computing enables users to use infrastructure and applications via the internet, without installing and maintaining them on-premises. In this regard, the cloud computing network 620 may include virtualized information technology (IT) infrastructure (e.g., servers 624-1-624-N, the data storage module 622, operating system software, networking, and other infrastructure) that is abstracted so that the infrastructure can be pooled and/or divided irrespective of physical hardware boundaries. In some embodiments, the cloud computing network 620 can provide users with services in the form of building blocks that can be used to create and deploy various types of applications in the cloud on a metered basis.

Various components of the cloud computing system 600 may be operable to implement the above operations in their entirety or contribute to the operations in part. Some embodiments disclosed herein may utilize instructions (e.g., code/software) accessible via a computer-readable storage medium for use by various components in the cloud computing system 600 to implement all or parts of the various operations disclosed hereinabove. Examples of such components include the computing systems 602-1-602-N.

Exemplary components of the computing systems 602-1-602-N may include at least one processor 604, a computer readable storage medium 614, program and data memory 606, input/output (I/O) devices 608, a display device interface 612, and a network interface 610. For the purposes of this description, the computer readable storage medium 614 comprises any physical media that is capable of storing a program for use by the computing system 602. For example, the computer-readable storage medium 614 may be an electronic, magnetic, optical, electromagnetic, infrared, semiconductor device, or other non-transitory medium. Examples of the computer-readable storage medium 614 include a solid-state memory, a magnetic tape, a removable computer diskette, a random access memory (RAM), a read-only memory (ROM), a rigid magnetic disk, and an optical disk. Some examples of optical disks include Compact Disk Read Only Memory (CD-ROM), Compact Disk-Read/Write (CD-R/W), Digital Versatile Disc (DVD), and Blu-Ray Disc.

The processor 604 is coupled to the program and data memory 606 through a system bus 616. The program and data memory 606 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 608 (including but not limited to keyboards, displays, touchscreens, microphones, pointing devices, etc.) may be coupled either directly or through intervening I/O controllers. Network adapter interfaces 610 may also be integrated with the system to enable the computing system 602 to become coupled to other computing systems or storage devices through intervening private or public networks. The network adapter interfaces 610 may be implemented as modems, cable modems, Small Computer System Interface (SCSI) devices, Fibre Channel devices, Ethernet cards, wireless adapters, etc. Display device interface 612 may be integrated with the system to interface to one or more display devices, such as screens for presentation of data generated by the processor 604.

What is claimed is:

1. A system for visualizing computed tomography angiography (CTA) images, comprising:
a data interface configured to receive CTA image data in a Digital Imaging and Communications in Medicine (DICOM) format; and
a processor configured to render a plurality of two-dimensional CTA slice images into a three-dimensional (3D) volumetric dataset from the CTA image data, to filter the 3D volumetric dataset to resolve vascular structures relative to surrounding tissue and bone, and to generate an extended reality (XR) holographic representation of the filtered 3D volumetric dataset; and
an XR display device configured to present the holographic representation to a user for the user to manipulate the holographic representation and identify a vascular abnormality.

2. The system of claim 1, wherein:
the processor comprises one or more graphical processing units (GPUs).

3. The system of claim 1, wherein:
the XR display device comprises one or more camera modules configured to detect gestures by the user to manipulate the holographic representation.

4. The system of claim 1, wherein:
the processor is further configured to filter the 3D volumetric dataset with a clipping tool to remove bone elements from the volumetric dataset to expose vascular structures.

5. The system of claim 1, wherein:
the processor is further configured to spotlight one or more vessels within the holographic representation with a probe implemented by a gesture of the user.

6. The system of claim 1, wherein:
the processor is further configured to apply one or more preset transfer functions to emphasize vascular structures relative to bone or soft tissue.

7. The system of claim 1, wherein:
the system is configured to facilitate detection of a large vessel occlusion in a cerebral artery.

8. The system of claim 1, wherein:
the processor is further configured to render the 3D volumetric dataset in substantially real-time, the rendering being completed within about one minute of receipt of the CTA image data.

9. The system of claim 1, wherein:
the processor is further configured to allow the user to adjust a transparency level of non-vascular structures within the 3D volumetric dataset.

10. The system of claim 1, wherein:
the processor is further configured to generate the holographic representation with one or more preset anatomical views including at least one of an axial orientation, a coronal orientation, or a sagittal orientation.

11. The system of claim 1, wherein:
the XR display device is further configured to present at least one holographic radiology image pinned in a spatial location relative to the holographic representation of the 3D volumetric dataset.

12. A method for visualizing computed tomography angiography (CTA) images, comprising:
receiving CTA image data in a Digital Imaging and Communications in Medicine (DICOM) format; and
rendering a plurality of two-dimensional CTA slice images into a three-dimensional (3D) volumetric dataset from the CTA image data;
filtering the 3D volumetric dataset to resolve vascular structures relative to surrounding tissue and bone;
generating an extended reality (XR) holographic representation of the filtered 3D volumetric dataset; and
presenting the holographic representation to a user via an XR display device configured to for the user to manipulate the holographic representation and identify a vascular abnormality.

13. The method of claim 12, wherein:
at least one of rendering, filtering, generating, or presenting is performed using one or more graphical processing units (GPUs).

14. The method of claim 12, further comprising:
detecting gestures by the user to manipulate the holographic representation via one or more camera modules configured with the XR display device.

15. The method of claim 12, wherein filtering further comprises:
filtering the 3D volumetric dataset with a clipping tool to remove bone elements from the volumetric dataset to expose vascular structures.

16. The method of claim 12, further comprising:
spotlighting one or more vessels within the holographic representation with a probe implemented by a gesture of the user.

17. The method of claim 12, further comprising:
applying one or more preset transfer functions to emphasize vascular structures relative to bone or soft tissue.

18. The method of claim 12, further comprising:
facilitating detection of a large vessel occlusion in a cerebral artery.

19. The method of claim 12, wherein:
rendering the 3D volumetric dataset is performed in substantially real-time, with the rendering being completed within about one minute of receipt of the CTA image data.

20. The method of claim 12, further comprising:
allowing the user to adjust a transparency level of non-vascular structures within the 3D volumetric dataset.

21. The method of claim 12, further comprising:
generating the holographic representation with one or more preset anatomical views including at least one of an axial orientation, a coronal orientation, or a sagittal orientation.

22. The method of claim 12, further comprising:
presenting at least one holographic radiology image pinned in a spatial location relative to the holographic representation of the 3D volumetric dataset via the XR display device.

23. A non-transitory computer readable medium comprising instructions that, when executed by a processing system, are operable to direct the processing system to visualize computed tomography angiography (CTA) images, the instructions further directing the processing system to:
receive CTA image data in a Digital Imaging and Communications in Medicine (DICOM) format; and
render a plurality of two-dimensional CTA slice images into a three-dimensional (3D) volumetric dataset from the CTA image data;
filter the 3D volumetric dataset to resolve vascular structures relative to surrounding tissue and bone;
generate an extended reality (XR) holographic representation of the filtered 3D volumetric dataset; and
present the holographic representation to a user via an XR display device configured to for the user to manipulate the holographic representation and identify a vascular abnormality.

24. The computer readable medium of claim 23, wherein:
at least one of rendering, filtering, generating, or presenting is performed using one or more graphical processing units (GPUs).

25. The computer readable medium of claim 23, the instructions further directing the processing system to:
detect gestures by the user to manipulate the holographic representation via one or more camera modules configured with the XR display device.

26. The computer readable medium of claim 23, wherein filtering further comprises:
filtering the 3D volumetric dataset with a clipping tool to remove bone elements from the volumetric dataset to expose vascular structures.

27. The computer readable medium of claim 23, the instructions further directing the processing system to:
spotlight one or more vessels within the holographic representation with a probe implemented by a gesture of the user.

28. The computer readable medium of claim 23, the instructions further directing the processing system to:
apply one or more preset transfer functions to emphasize vascular structures relative to bone or soft tissue.

29. The computer readable medium of claim 23, the instructions further directing the processing system to:
facilitate detection of a large vessel occlusion in a cerebral artery.

30. The computer readable medium of claim 23, wherein:
rendering the 3D volumetric dataset is performed in substantially real-time, with the rendering being completed within about one minute of receipt of the CTA image data.

31. The computer readable medium of claim 23, the instructions further directing the processing system to:
allow the user to adjust a transparency level of non-vascular structures within the 3D volumetric dataset.

32. The computer readable medium of claim 23, the instructions further directing the processing system to:
generate the holographic representation with one or more preset anatomical views including at least one of an axial orientation, a coronal orientation, or a sagittal orientation.

33. The computer readable medium of claim 23, the instructions further directing the processing system to:
present at least one holographic radiology image pinned in a spatial location relative to the holographic representation of the 3D volumetric dataset via the XR display device.
